(19) United States
(12) Patent Application Publication
(10) Pub. No.: US 2014/0342388 A1
(43) Pub. Date: Nov. 20, 2014

(54) REAGENT FOR LABELING PRIMARY AMINE GROUPS IN PROTEINS

(71) Applicants: Yang Cai, New Orleans, LA (US); Matthew A. Tarr, Metairie, LA (US); Ujwal S. Patil, New Orleans, LA (US); Haiou Qu, Little Rock, AR (US); Daniela Caruntu, Mount Pleasant, MI (US); Charles J. O'Connor, New Orleans, LA (US)

(72) Inventors: Yang Cai, New Orleans, LA (US); Matthew A. Tarr, Metairie, LA (US); Ujwal S. Patil, New Orleans, LA (US); Haiou Qu, Little Rock, AR (US); Daniela Caruntu, Mount Pleasant, MI (US); Charles J. O'Connor, New Orleans, LA (US)

(21) Appl. No.: 14/229,911

(22) Filed: Mar. 29, 2014

Related U.S. Application Data
(60) Provisional application No. 61/808,252, filed on Apr. 4, 2013.

(51) Int. Cl. G01N 33/58; G01N 33/68

(52) U.S. Cl. CPC G01N 33/585; G01N 33/6842; G01N 33/6848; G01N 2333/705
USPC 435/29; 436/89

(57) ABSTRACT

A reagent includes a magnetic nanoparticle, a cleavable linker, and a primary amine reactive group. A first end of the cleavable linker may conjugate to a surface of the magnetic nanoparticle. A second end of the cleavable linker may conjugate to a primary amine reactive group. A method of using the reagent includes adding the reagent to a native protein mixture such that magnetic nanoparticles may conjugate to the protein with exposed primary amine groups in the native protein mixture. The method also includes separating the proteins with exposed primary amine group from other components of the native protein mixture under a magnetic field. The method further includes removing the magnetic nanoparticles from the proteins with the exposed primary amine groups from at cleavable linkers. The method finally includes identifying sequences and/or sites of the exposed primary amine groups in the proteins.

This application claims the benefit of U.S. Provisional Application No. 61/808,252, filed Apr. 4, 2013.

### I. FIELD

The present disclosure is generally related to a reagent for labeling exposed primary amine groups in proteins.

### II. DESCRIPTION OF RELATED ART

A microbial pathogen generally attacks a cell through proteins located on a cell surface. Host immune system may launch defense by identifying the surface proteins. Since the surface proteins are often co-precipitated with proteins from other cell compartments, it is difficult to isolate a pure fraction of the surface proteins for further study. In addition, since exposed portions of the surface proteins may be locations from which the host immune system initiates defense against the invasion of the microbial pathogen, it is important to determine which portions of the surface proteins are exposed.

Magnetic nanoparticles (NP), such as iron oxide (Fe₃O₄) NP, have been used to separate proteins with exposed primary amine groups from other components. The NP may be first coated with silica (SiO₂) to prevent oxidation of the NP, to prevent interactions between the NP and the proteins, and to facilitate surface modification by a first functional group, such as amine (—NH₂) or thiol (—SH) functional group. The NP may further conjugate to a second functional group (i.e., a primary amine reactive group), such as a N-hydroxysuccinimidyl (NHS) ester functional group or a sulfo-NHS ester functional group. The NP conjugating to the NHS ester functional group may form covalent bonds with the proteins with the exposed primary amine groups. Since the NP may not be removed from the proteins with the exposed primary amine groups once the NP conjugate to the proteins, currently technology is unable to identify sequences and sites of the exposed primary amine groups in the proteins.

### III. SUMMARY

This disclosure presents particular embodiments of a reagent that includes a magnetic nanoparticle, such as an iron oxide (Fe₃O₄) super paramagnetic nanoparticle, whose surface may conjugate to a first end of a cleavable linker (e.g., a disulfide cleavable linkers, a tertiary carbamate cleavable linker, or a beta keto ester cleavable linker). A second end of the cleavable linker may conjugate to a primary amine reactive functional group, such as a N-hydroxysuccinimidyl (NHS) ester functional group or a sulfo-NHS ester functional group. When the reagent is added to a native protein mixture, magnetic nanoparticles may conjugate to segments of a protein with exposed primary amine groups. The magnetic nanoparticles conjugating to the segments of the protein with the exposed primary amine groups may be separated from other components using a magnet. The magnetic nanoparticles may be removed from the segments of the protein with the exposed primary amine groups at cleavable linkers. A mass spectrometry analysis may be performed to identify sequences and sites of the exposed primary amine groups in the segments of the protein.

In a particular embodiment, a reagent includes a magnetic nanoparticle, such as an Fe₃O₄ super paramagnetic nanoparticle. The magnetic nanoparticle may be coated with a coating layer, such as a silica (SiO₂) coating layer, to prevent the magnetic nanoparticle from oxidation, to prevent an interaction between the magnetic nanoparticle and a protein, and to facilitate surface modification by a functional group (e.g., a primary amine functional group). A surface of the magnetic nanoparticle may conjugate to a first end of a cleavable linker. A second end of the cleavable linker may conjugate to a primary amine reactive group, such as a NHS ester functional group or a sulfo-NHS ester functional group.

In another particular embodiment, a method of synthesizing the reagent includes performing one or more chemical reactions on a surface of the magnetic nanoparticle. As a result, a cleavable linker may be formed on the surface of the magnetic nanoparticle with a first end of a cleavable linker conjugating to the surface of the magnetic nanoparticle, and a second end of the cleavable linker conjugating to a primary amine reactive group, such as a NHS ester functional group or a sulfo-NHS ester functional group.

In another particular embodiment, a method of using the reagent to identify sequences and sites of exposed primary amine groups in a protein includes adding the reagent to a native protein mixture. The native protein mixture may include the protein with the exposed primary amine groups. As a result, magnetic nanoparticles of the reagent conjugate to the protein with the exposed primary amine groups. The method also includes separating the protein with the exposed primary amine groups from other components using a magnet. The method further includes removing the magnetic nanoparticles from the protein with the exposed primary amine groups at cleavable linkers. The method finally includes performing a mass spectrometry analysis to identify sequences and sites of the exposed primary amine groups in the protein.

One particular advantage provided by at least one of the disclosed embodiments is that the reagent enables separation of the protein with the exposed primary amine groups from other components. Thus, the protein with the exposed primary amine groups may be efficiently isolated for further study.

Another particular advantage provided by at least one of the disclosed embodiments is that the reagent enables formation of the cleavable linkers on the surfaces of the magnetic nanoparticles. Thus, the magnetic nanoparticles may be removed from the protein with the exposed primary amine groups at the cleavable linkers, and the sequences and sites of the exposed primary amine groups in the protein may be identified.

Another particular advantage provided by at least one of the disclosed embodiments is that the reagent enables labeling the exposed primary amine groups of the protein in aqueous solution and at/below room temperature. Thus, native structures of the protein may be preserved because cells are alive during the separation and labeling.

Another particular advantage provided by at least one of the disclosed embodiments is that the magnetic nanoparticles of the reagent are too large to penetrate cell surfaces when the reagent conjugates to surface proteins on cells. Thus, cells may not be damaged by the magnetic nanoparticles and intact cell structures may be preserved.

Other aspects, advantages, and features of the present disclosure will become apparent after review of the entire application, including the following sections: Brief Description of the Drawings, Detailed Description, and the Claims.

### IV. BRIEF DESCRIPTION OF THE DRAWINGS

FIG. 1 is a diagram of a particular embodiment of a reagent that includes a magnetic nanoparticle, a cleavable linker, and a primary amine reactive group;

FIG. 2 is a diagram showing chemical structures of particular embodiments of a cleavable linker;

FIG. 3 is a diagram showing chemical structures of particular embodiments of a primary amine reactive group;

FIG. 4 is a diagram of a particular embodiment of forming an iron oxide (Fe₃O₄) super paramagnetic nanoparticle;

FIG. 5 is a diagram of a particular embodiment of forming a silica (SiO₂) coating layer on a surface of a magnetic nanoparticle;

FIG. 6 is a diagram of a particular embodiment of modifying a magnetic nanoparticle with an amine functional group;

FIG. 7 is a diagram of a particular embodiment of conjugating a magnetic nanoparticle modified with an amine functional group to a NHS ester functional group to form a reagent that includes a disulfide cleavable linker;

FIG. 8 is a diagram of a particular embodiment of modifying a magnetic nanoparticle with a thiol functional group;

FIG. 9 is a diagram of a particular embodiment of conjugating a magnetic nanoparticle modified with a thiol functional group to a NHS ester functional group to form a reagent that includes a disulfide cleavable linker;

FIG. 10 is a diagram of a particular embodiment of conjugating a reagent to an exposed primary amine group in a protein;

FIG. 11 is a diagram of a particular embodiment of removing a magnetic nanoparticle from a protein with an exposed primary amine group conjugating to a reagent at a cleavable linker;

FIG. 12 is a flow chart of a particular embodiment of a method of forming a reagent that is modified with a functional group; and

FIG. 13 is a flow chart of a particular embodiment of a method of using a reagent to identify sequences and sites of exposed primary amine groups in a protein.

### V. DETAILED DESCRIPTION

This disclosure relates generally to a reagent that includes a magnetic nanoparticle, such as an iron oxide (Fe₃O₄) super paramagnetic nanoparticle, a cleavable linker, and a primary amine reactive group. A surface of the magnetic nanoparticle may conjugate to a first end of a cleavable linker (e.g., a disulfide bond cleavable linker, a tertiary carbamate cleavable linker, or a beta keto ester cleavable linker). A second end of the cleavable linker may conjugate to a primary amine reactive group, such as a N-hydroxysuccinimidyl (NHS) ester functional group or a sulfo-NHS ester functional group. The reagent may enable an identification of sequences and sites of exposed lysine side chains in a protein.

A method of forming the reagent includes performing one or more chemical reactions on the surface of the magnetic nanoparticle. As a result, the cleavable linker may be formed on the surface of the magnetic nanoparticle with the first end of a cleavable linker conjugating to the surface of the magnetic nanoparticle, and the second end of the cleavable linker conjugating to the primary amine reactive group.

A method of using the reagent includes adding the reagent to a native protein mixture. The native protein mixture may include the protein with the exposed primary amine groups. As a result, magnetic nanoparticles of the reagent may conjugate to the protein with the exposed primary amine groups. The method also includes separating the protein with the exposed primary amine groups from other components under a magnetic field. The method further includes removing the magnetic nanoparticles from the protein with the exposed primary amine groups at cleavable linkers. The method finally includes identifying sequences and sites of the exposed primary amine groups in the protein.

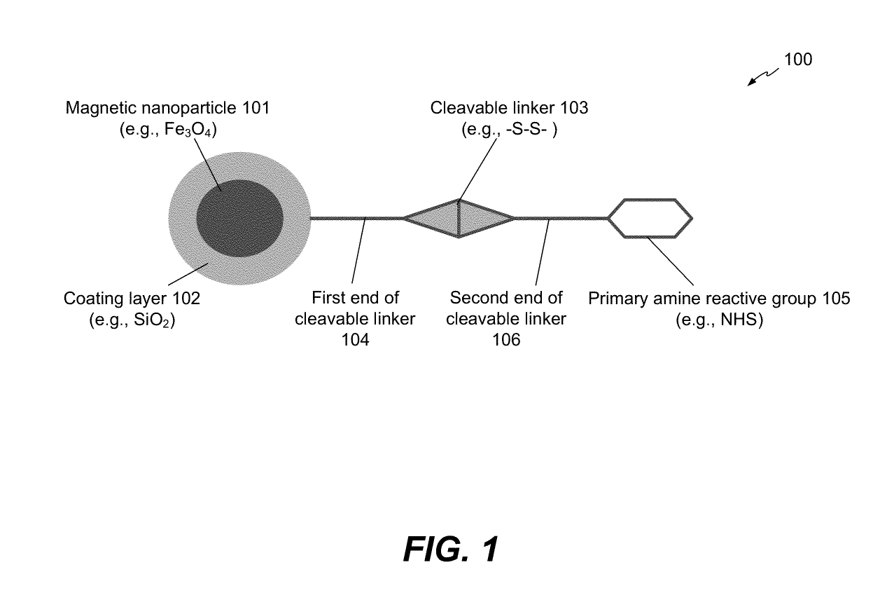

Referring to FIG. 1, a diagram of a particular embodiment of a reagent that includes a magnetic nanoparticle, a cleavable linker, and a primary amine reactive group is disclosed and generally designated 100. In FIG. 1, for purpose of illustration, the reagent 100 may include a magnetic nanoparticle (e.g., a Fe₃O₄ super paramagnetic nanoparticle) 101, a coating layer (e.g., a SiO₂ coating layer) 102, a cleavable linker (e.g., a disulfide bond cleavable linker, a tertiary carbamate cleavable linker, or a beta keto ester cleavable linker) 103, and a primary amine reactive group (e.g., a NHS ester functional group or a sulfo-NHS ester functional group) 105. The magnetic nanoparticle 101 may be coated with the coating layer 102. A first end 104 of the cleavable linker 103 may conjugate to the magnetic nanoparticle 101. A second end 106 of the cleavable linker 103 may conjugate to the primary amine reactive group 105. The reagent 100 may enable an identification of sequences and sites of exposed primary amine groups in a protein.

The reagent 100 includes a magnetic nanoparticle 101. An example of the magnetic nanoparticle 101 may include an Fe₃O₄ super paramagnetic nanoparticle. The Fe₃O₄ super paramagnetic nanoparticle may be produced by a conventional co-precipitation method.

A surface of the magnetic nanoparticle 101 may be coated with a coating layer 102. An example of the coating layer 102 may include a SiO₂ coating layer. When the magnetic nanoparticle includes an Fe₃O₄ super paramagnetic nanoparticle, the coating layer 102 may prevent the Fe₃O₄ super paramagnetic nanoparticle from further oxidation. In addition, the coating layer 102 may prevent an interaction between the magnetic nanoparticle and proteins to be labeled. Furthermore, the coating layer 102 may facilitate surface modification by a functional group (e.g., an amine functional group or a thiol group). The coating layer 102 may be prepared by a sol-gel method.

The reagent 100 includes a cleavable linker 103. A first end 104 of the cleavable linker 103 may conjugate to the surface of the magnetic nanoparticle 101. Examples of the cleavable linker 103 may include a disulfide bond cleavable linker, a tertiary carbamate cleavable linker, and a beta keto ester cleavable linker, as further described below. When the reagent 100 is configured to conjugate to a protein with exposed primary amine groups, the magnetic nanoparticle 101 may be removed from the protein with the exposed primary amine groups at the cleavable linker 103 such that sequences and sites of the exposed primary amine groups may be identified.

The reagent 100 includes a primary amine reactive group 105. The primary amine reactive group 105 may conjugate to a second end 106 of the cleavable linker 103. Examples of the primary amine reactive group 105 may include a NHS ester functional group and a sulfo-NHS ester functional group. The primary amine reactive group may be configured to conjugate to an exposed primary amine group of a protein.

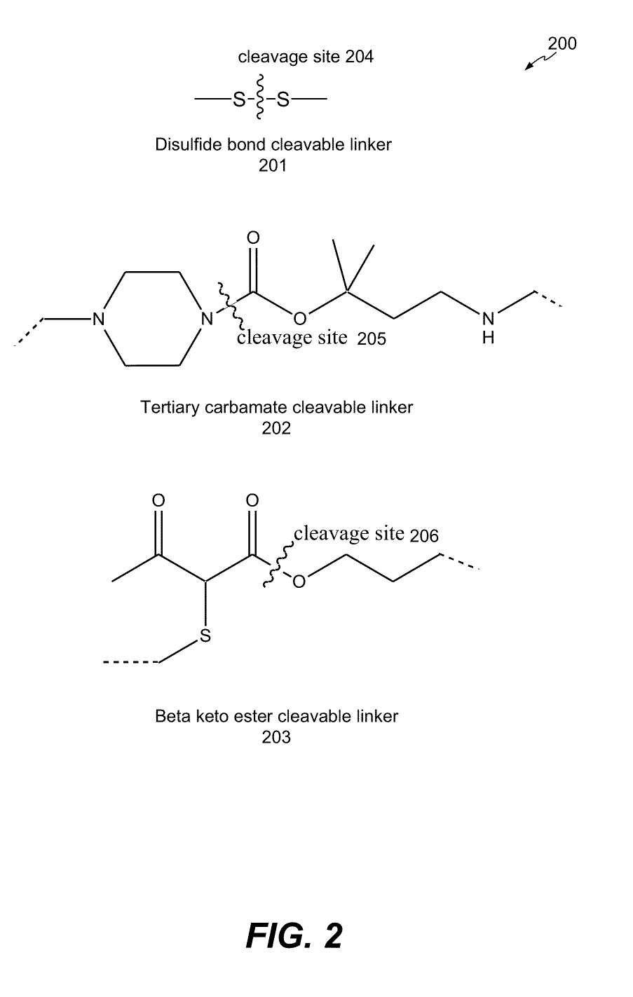

Referring to FIG. 2, a diagram showing chemical structures of particular embodiments of a cleavable linker is disclosed and designated 200. The cleavable linker 200 (e.g., the cleavable linker 103 of FIG. 1) may be formed on a surface of a magnetic nanoparticle (e.g., the magnetic nanoparticle 101 of FIG. 1) by performing one or more chemical reactions as further described below. As a result, a first end (e.g., the first end 104 of FIG. 1) of the cleavable linker 200 may conjugate to the surface of the magnetic nanoparticle, and a second end (e.g., the second end 106 of FIG. 1) of the cleavable linker 200 may conjugate to a primary amine reactive group (e.g., the primary amine reactive group 105 of FIG. 1). Examples of the cleavable linker 200 may include a disulfide bond cleavable linker 201, a tertiary carbamate cleavable linker 202, and a beta keto ester cleavable linker 203. The cleavable linker 200 may be cleaved. For example, the disulfide bond cleavable linker 201 may be cleaved at a cleavage site 204. As another example, the tertiary carbamate cleavable linker 202 may be cleaved at a cleavage site 205. As a further example, the beta keto ester cleavable linker 203 may be cleaved at a cleavage site 206. When a reagent (e.g., the reagent 100 of FIG. 1) is configured to conjugate to a protein with exposed primary amine groups, magnetic nanoparticles may be removed from the protein with the exposed primary amine groups at the cleavage sites (e.g., the cleavable sites 204-206) of the cleavable linkers 200 such that a mass spectrometry analysis may be performed to identify sequences and sites of the exposed primary amine groups in the protein.

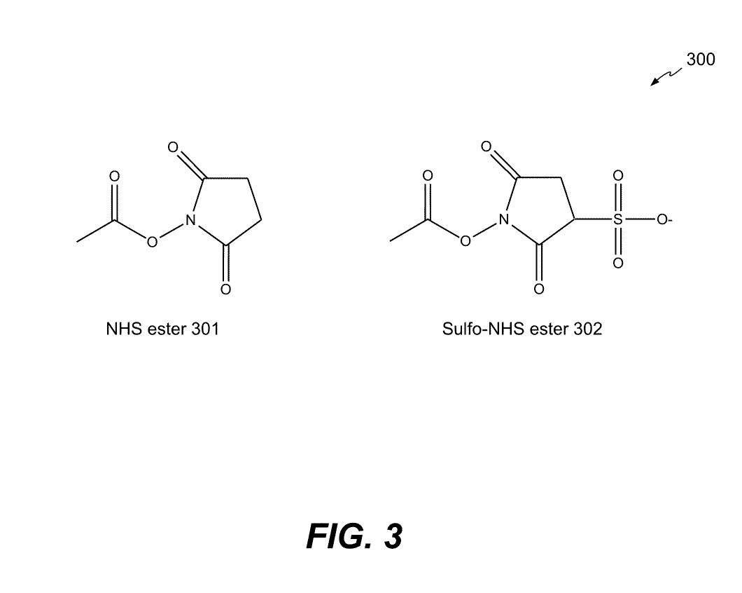

Referring to FIG. 3, a diagram showing chemical structures of particular embodiments of a primary amine reactive group is disclosed and generally designated 300. The primary amine reactive group 300 (e.g., the primary amine reactive group 105 of FIG. 1) may conjugate to a cleavable linker (e.g., the cleavable linker 103 of FIG. 1 and 200 of FIG. 2) by performing one or more chemical reactions as further described below. Examples of the primary amine reactive group 300 may include a NHS ester functional group 301 and a sulfo-NHS ester function group 302. The primary amine reactive group 300 may form a convalent bond with an exposed primary amine group of a protein such that a mass spectrometry analysis may be performed to identify sequences and sites of the exposed lysine side chains in the protein.

FIGS. 1-3 thus illustrate a reagent 100. The reagent 100 may include a magnetic nanoparticle 101, a coating layer 102, a cleavable linker 103 and 200, and a primary amine reactive group 105 and 300. The reagent 100 may enable an identification of sequences and sites of exposed primary amine groups in a protein.

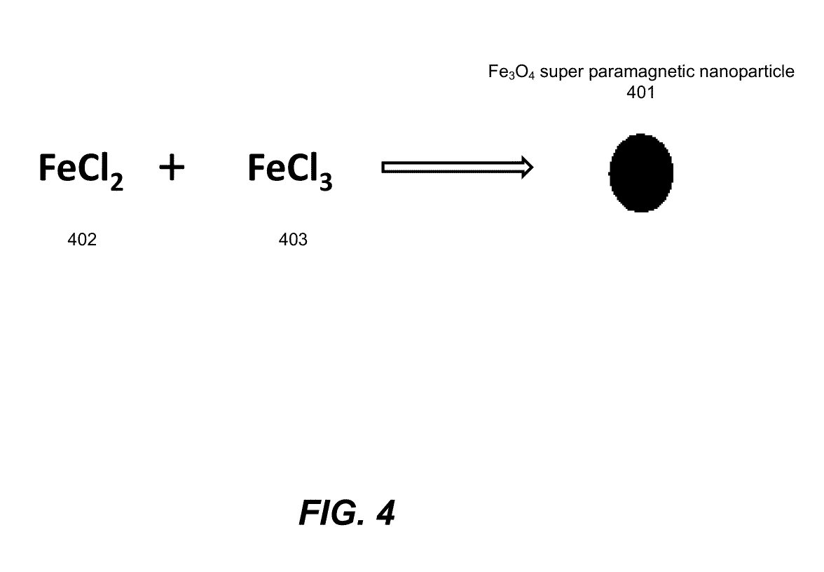

Referring to FIG. 4, a diagram of a particular embodiment of forming an Fe₃O₄ super paramagnetic nanoparticle is shown. The Fe₃O₄ super paramagnetic nanoparticle 401 may be formed by a conventional co-precipitation method. In one embodiment, iron (II) chloride tetrahydrate (FeCl₂.4H₂O) 402 and iron (III) chloride hexahydrate (FeCl₃.6H₂O) 403 may be dissolved in deionized water under nitrogen (N₂) with vigorous stirring at 90° C. Aqueous ammonia may be added to the solution. As a result of a reaction, Fe₃O₄ super paramagnetic nanoparticles may be formed. The Fe₃O₄ super paramagnetic nanoparticles may be separated from the solution using a magnet.

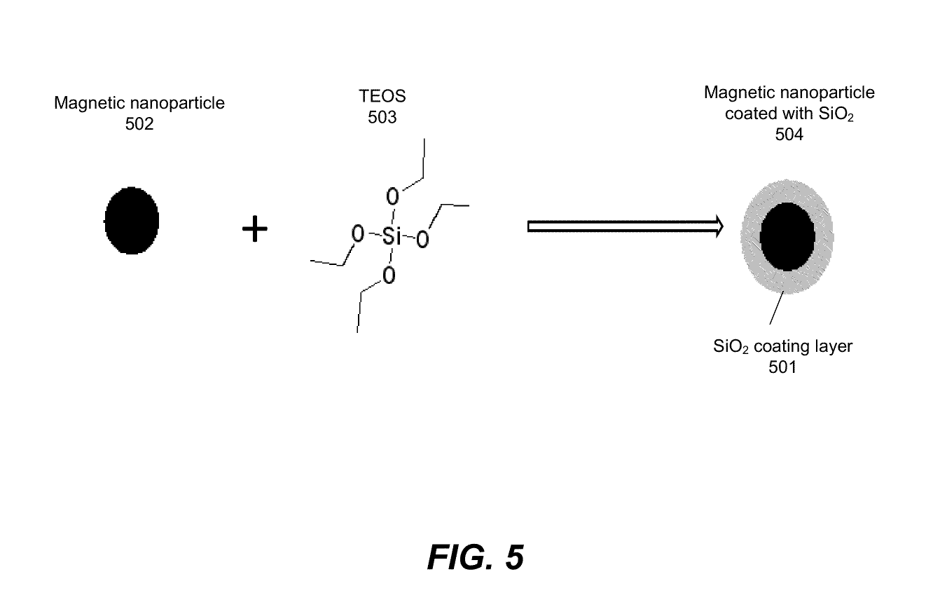

Referring to FIG. 5, a diagram of a particular embodiment of forming a SiO₂ coating layer on a surface of a magnetic nanoparticle is shown. The SiO₂ coating layer 501 may be formed by a sol-gel method. In one embodiment, magnetic nanoparticles 502 may be dispersed in a mixture of ethanol, ultrapure water, and ammonia under ultrasonication. Tetraethylorthosilicate (TEOS) 503 may be added to the solution. As a result of a reaction, magnetic nanoparticles coated with SiO₂ 504 may be formed. The magnetic nanoparticles coated with SiO₂ 504 may be separated from the solution using a magnet.

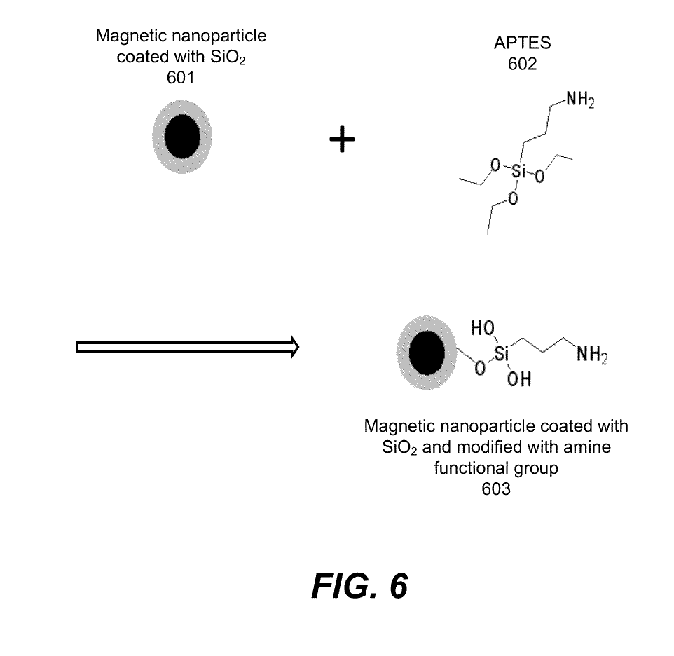

Referring to FIG. 6, a diagram of a particular embodiment of modifying a magnetic nanoparticle with an amine functional group is shown. Magnetic nanoparticles coated with SiO₂ 601 (e.g., the magnetic nanoparticles coated with SiO₂ 504 of FIG. 5) may be dispersed in a mixture of DMF and toluene under ultrasonication. 3-aminopropyltriethoxysilane (APTES) 602 may be added to the solution. As a result of a reaction, magnetic nanoparticles modified with the amine functional group 603 may be formed. The magnetic nanoparticles modified with the amine functional group 603 may be separated from the solution using a magnet.

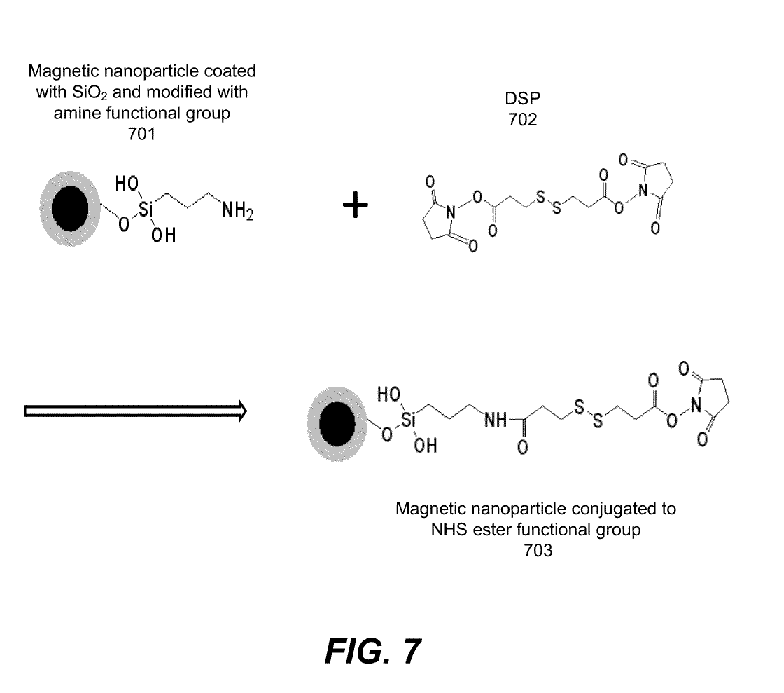

Referring to FIG. 7, a diagram of a particular embodiment of conjugating a magnetic nanoparticle modified with an amine functional group to a NHS ester functional group to form a reagent that includes a disulfide cleavable linker is shown. Magnetic nanoparticles modified with an amine functional group 701 may be dispersed in ethanol under sonication. Dithiobis(succinimidylpropionate) (DSP) 702 may be added to the solution. As a result of a reaction, magnetic nanoparticles conjugating to a NHS ester functional group 703 may be formed. The magnetic nanoparticles conjugating to a NHS ester functional group 703 may be separated from the solution using a magnet.

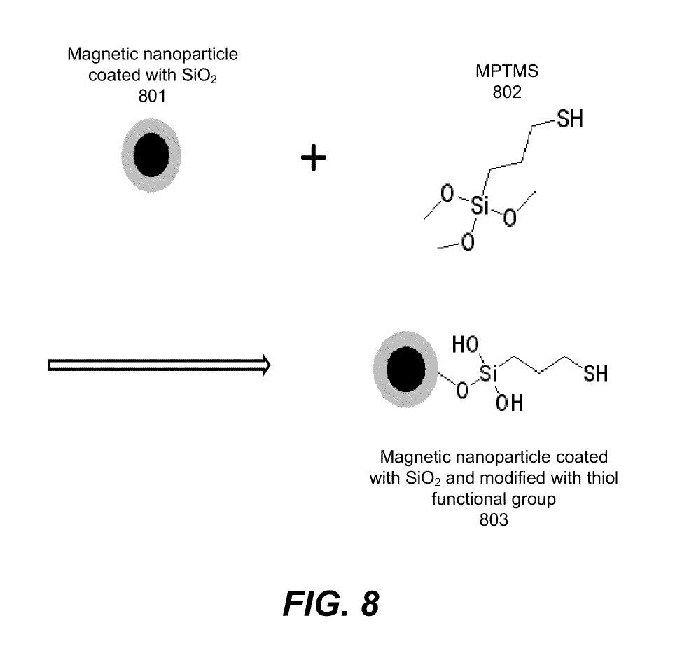

Referring to FIG. 8, a diagram of a particular embodiment of modifying a magnetic nanoparticle with a thiol functional group is shown. The magnetic nanoparticles coated with SiO₂ (e.g., the magnetic nanoparticles coated with SiO₂ 504 of FIG. 5) 801 may be dispersed in a mixture of DMF and toluene under ultrasonication. 3-mercaptopropyltrimethoxysilane (MPTMS) 802 may be added to the solution. As a result of a reaction, magnetic nanoparticles modified with a thiol functional group 803 may be formed. The magnetic nanoparticles modified with a thiol functional group 803 may be separated from the solution by a magnet.

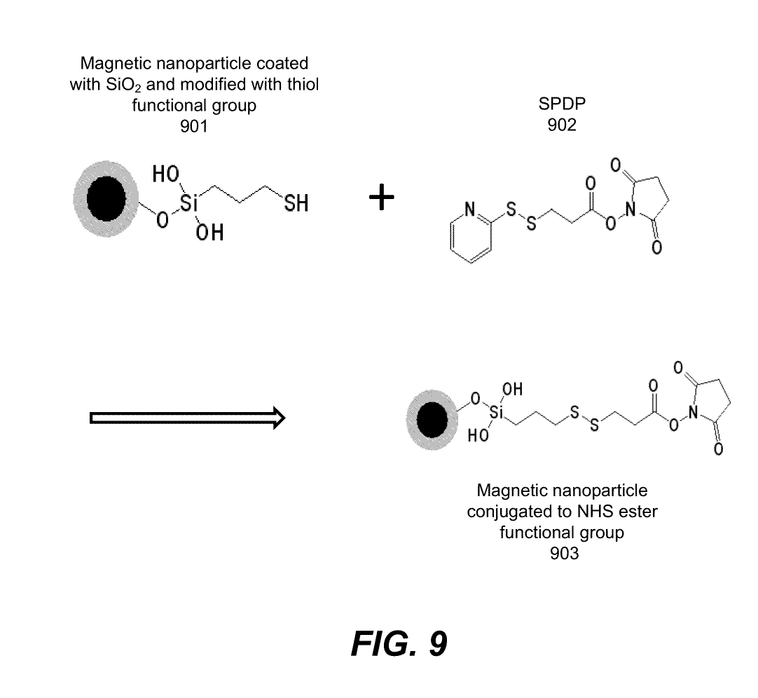

Referring to FIG. 9, a diagram of a particular embodiment of conjugating a magnetic nanoparticle to a NHS ester functional group to form a reagent that includes a cleavable linker is shown. Magnetic nanoparticles modified with a thiol functional group 901 (e.g., the magnetic nanoparticles modified with a thiol functional group 803 of FIG. 8) may be dispersed in ethanol under sonication. N-Succinimidyl 3-(2-pyridyldithio)-propionate (SPDP) 902 may be added to the solution. As a result of a reaction, magnetic nanoparticles conjugating to a NHS ester functional group 903 may be formed. The magnetic nanoparticles conjugating to a NHS ester functional group 903 may be separated from the solution using a magnet.

FIGS. 4-9 thus illustrate methods of forming a reagent 100. In one embodiment, the method may include forming a magnetic nanoparticle (FIG. 4), forming a coating layer (FIG. 5) on a surface of the magnetic nanoparticle, forming a magnetic nanoparticle modified with an amine functional group (FIG. 6), and forming a magnetic nanoparticle conjugating to a NHS ester functional group (FIG. 7). In another embodiment, the method may include forming a magnetic nanoparticle (FIG. 4), forming a coating layer on a surface of the magnetic nanoparticle (FIG. 5), forming a magnetic nanoparticle modified with a thiol functional group (FIG. 8), and forming a magnetic nanoparticle conjugating to a NHS ester functional group (FIG. 9). The reagent 100 may enable an identification of sequences and sites of exposed primary amine groups in a protein.

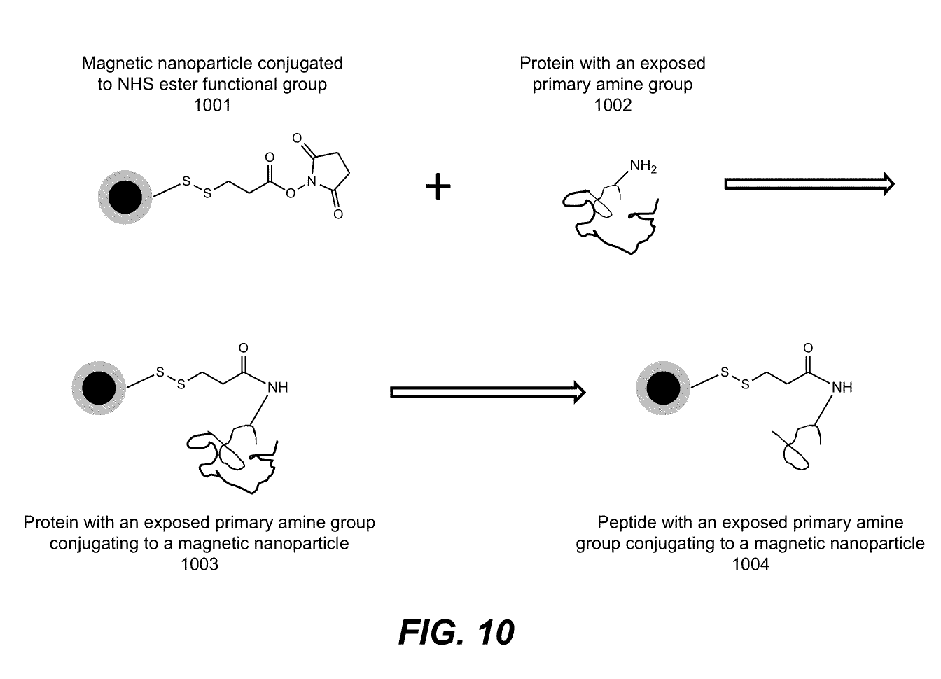

Referring to FIG. 10, a diagram of a particular embodiment of conjugating a reagent to exposed primary amine groups of a protein is shown. A solution that includes a native protein mixture may be added to a reagent 1001 (e.g., the reagent 100 of FIG. 1, 703 of FIG. 7, and 903 of FIG. 9) with continuous stirring. The native protein mixture may include a protein with exposed primary amine groups. Examples of the protein with exposed primary groups may include a protein with an exposed primary amine group 1002. The reaction may last for a specific period of time at room temperature. As a result of the reaction, the protein with the exposed primary amine groups may conjugate to the reagent. Examples of the protein with the exposed primary amine groups conjugating to the reagent may include the protein with the exposed primary amine group conjugating to the reagent 1003. The protein with the exposed primary amine groups conjugating to the reagent may be separated from other components in the mixture using a magnet. A chemical/enzymatic proteolysis process may be performed on the protein with the exposed primary amine groups to form segments of the protein with the exposed primary amine groups. Examples of segments of the protein with the exposed primary amine groups may include peptides with the exposed primary amine groups, such as a peptide with an exposed primary amine group 1004.

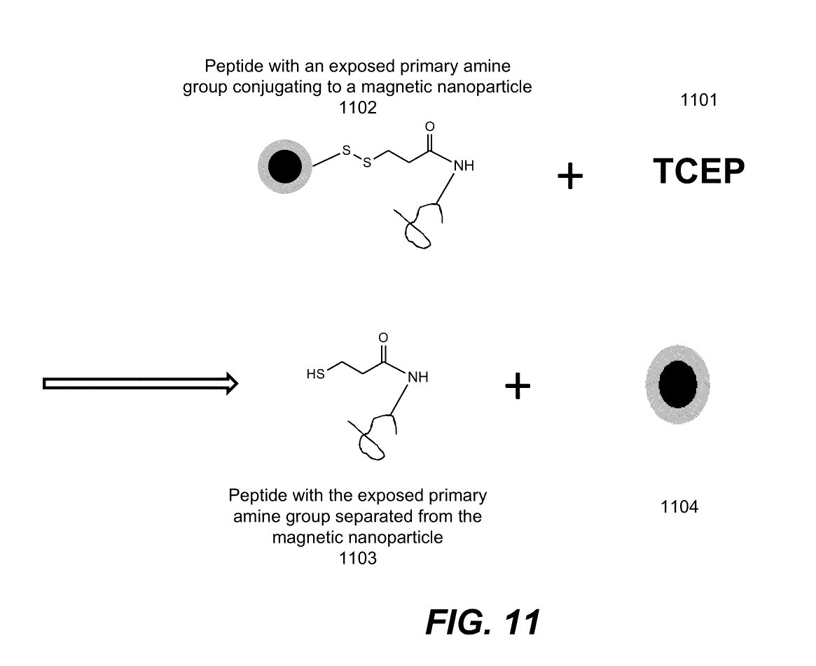

Referring to FIG. 11, a diagram of a particular embodiment of removing a magnetic nanoparticle from a segment of a protein with an exposed primary amine group conjugating to a reagent at a cleavable linker is shown. Tris(2-carboxyethyl)phosphine (TCEP) 1101 may be added to the segments of the protein with exposed primary amine groups conjugating to the reagent. Examples of the segments of the protein with exposed primary amine groups may include a peptide with an exposed primary group 1102 (e.g., the peptide with an exposed primary amine group 1104 of FIG. 10). The mixture may be incubated for a specific period of time at room temperature. As a result, the segments of the protein with exposed primary amine groups (e.g., the peptide with the exposed primary amine group 1103) may be separated from the magnetic nanoparticles (e.g., the magnetic nanoparticle 1104) at the cleavable linkers (e.g., the cleavable linkers 103 of FIG. 1 and 200 of FIG. 2). The magnetic nanoparticles may be removed from the segments of the protein with the exposed primary amine groups using a magnet. A mass spectrometry analysis may be performed on the segments of the protein with the exposed primary amine groups such that sequences and sites of the exposed primary amine groups in the protein may be identified.

FIGS. 10 and 11 thus illustrate a method of using a reagent (e.g., the reagent 100 of FIG. 1, 703 of FIG. 7, and 903 of FIG. 9). The method includes conjugating the reagent to a protein with exposed primary amine groups (FIG. 10). The method also includes removing magnetic nanoparticles from segments of the protein with the exposed primary amine groups at cleavable linkers (FIG. 11). The method finally includes performing a mass spectrometry analysis on the segments of the proteins with the exposed primary amine groups. The method may enable an identification of sequences and sites of the exposed primary amine groups in a native protein.

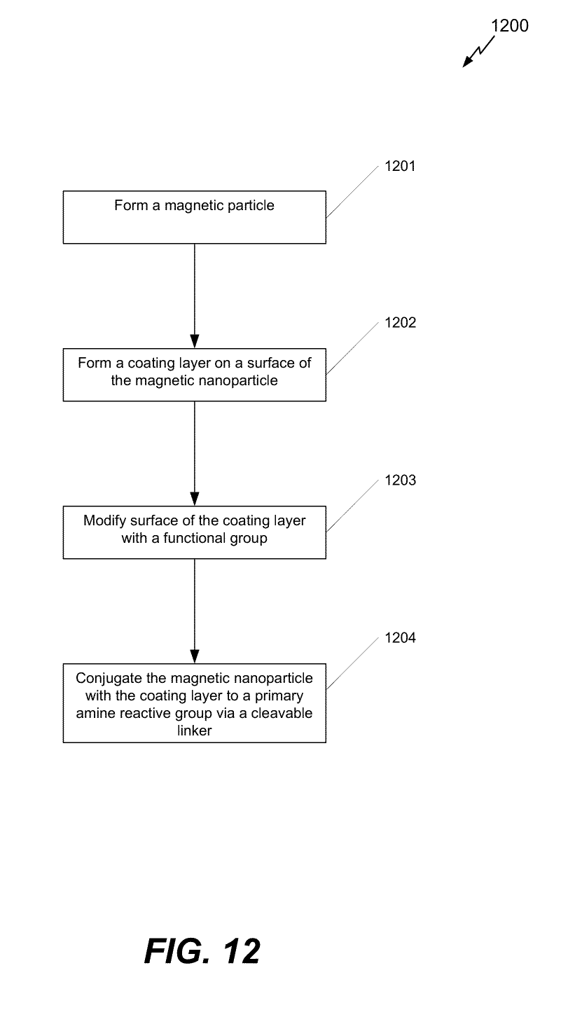

Referring to FIG. 12, a flow chart of a particular embodiment of a method of forming a reagent that is modified with a functional group is disclosed and generally designated 1200. The method may also be illustrated with reference to FIGS. 4-9.

At 1201, a magnetic nanoparticle (e.g., the Fe₃O₄ super paramagnetic nanoparticle 401 of FIG. 4) may be formed. For example, the Fe₃O₄ super paramagnetic nanoparticle 401 may be formed by a conventional co-precipitation method. In one embodiment, iron (II) chloride tetrahydrate (FeCl₂.4H₂O) 402 and iron (III) chloride hexahydrate (FeCl₃.6H₂O) 403 may be dissolved in deionized water under nitrogen (N₂) with vigorous stirring at 90° C. Aqueous ammonia may be added to the solution. As a result of a reaction, the Fe₃O₄ super paramagnetic nanoparticle 401 may be formed. The Fe₃O₄ super paramagnetic nanoparticles 401 may be separated from the solution using a magnet.

At 1202, a coating layer (e.g., the SiO₂ coating layer 501 of FIG. 5) may be formed on a surface of the magnetic nanoparticle. For example, the SiO₂ coating layer 501 may be formed by a sol-gel method. In one embodiment, the Fe₃O₄ super paramagnetic nanoparticles 502 may be dispersed in a mixture of ethanol, ultrapure water, and ammonia under ultrasonication. TEOS 503 may be added the mixture. As a result of a reaction, Fe₃O₄ super paramagnetic nanoparticles coated with SiO₂ 504 may be formed. The Fe₃O₄ super paramagnetic nanoparticles coated with SiO₂ 504 may be separated from the solution using a magnet.

At 1203, a magnetic nanoparticle coated with a coating layer (e.g., the Fe₃O₄ super paramagnetic nanoparticles coated with SiO₂ 601 of FIG. 6 and 801 of FIG. 8) may be modified with a functional group. In one embodiment, the Fe₃O₄ super paramagnetic nanoparticles coated with SiO₂ 601 may be dispersed in a mixture of DMF and toluene under ultrasonication. 3-aminopropyltriethoxysilane (APTES) 602 may be added to the solution. As a result of a reaction, The Fe₃O₄ super paramagnetic nanoparticles modified with an amine functional group 603 may be formed. The Fe₃O₄ super paramagnetic nanoparticles modified with the amine functional group 603 may be separated from the solution using a magnet.

In another embodiment, the Fe₃O₄ super paramagnetic nanoparticles coated with SiO₂ 801 may be dispersed in a mixture of DMF and toluene under ultrasonication. 3-mercaptopropyltrimethoxysilane (MPTMS) 802 may be added to the solution. As a result of a reaction, the Fe₃O₄ super paramagnetic nanoparticles modified with a thiol functional group 803 may be formed. The Fe₃O₄ super paramagnetic nanoparticles coated modified with a thiol functional group 803 may be separated from the solution using a magnet.

At 1204, a magnetic nanoparticle (e.g., the Fe₃O₄ super paramagnetic nanoparticles that are coated with SiO₂ and modified with a functional group 701 of FIG. 7 and 901 of FIG. 9) may conjugate to a NHS ester functional group or a sulfo-NHS ester functional group to form the reagent (e.g., the reagent 100 of FIG. 1, 703 of FIG. 7, and 903 of FIG. 9). In one embodiment, the Fe₃O₄ super paramagnetic nanoparticles that are coated with SiO₂ and modified with an amine functional group 701 may be dispersed in ethanol under sonication. DSP 702 may be added to the solution. The solution may be stirred. As a result of a reaction, the Fe₃O₄ super paramagnetic nanoparticles may conjugate to NHS ester functional groups 703. The Fe₃O₄ super paramagnetic nanoparticles conjugating to NHS ester functional groups 703 may be separated using a magnetic.

In another embodiment, the Fe₃O₄ super paramagnetic nanoparticles that are coated with SiO₂ and modified with a thiol functional group 901 may be dispersed in ethanol under sonication. SPDP 902 may be added to the solution. The solution may be stirred. As a result of a reaction, the Fe₃O₄ super paramagnetic nanoparticles may conjugate to NHS ester functional groups 903. The Fe₃O₄ super paramagnetic nanoparticles conjugating to NHS ester functional groups 903 may be separated using a magnet.

FIG. 12 thus illustrates a method of forming a reagent (e.g., the reagent 100 of FIG. 1, 703 of FIG. 7, and 903 of FIG. 9). The method may include forming a magnetic nanoparticle (e.g., the Fe₃O₄ super paramagnetic nanoparticle 401 of FIG. 4), forming a coating layer (e.g., the SiO₂ coating layer 501 of FIG. 5) on a surface of the magnetic nanoparticle, modifying the magnetic nanoparticle with a functional group (e.g., an amine functional group 603 of FIG. 6 or a thiol functional group 803 of FIG. 8), and conjugating the magnetic nanoparticle with the coating layer to a primary amine reactive group (e.g., the NHS ester functional group 703 of FIG. 7 and 903 of FIG. 9 or the sulfo-NHS ester functional group). The reagent may enable an identification of sequences and sites of the exposed primary amine groups in a protein.

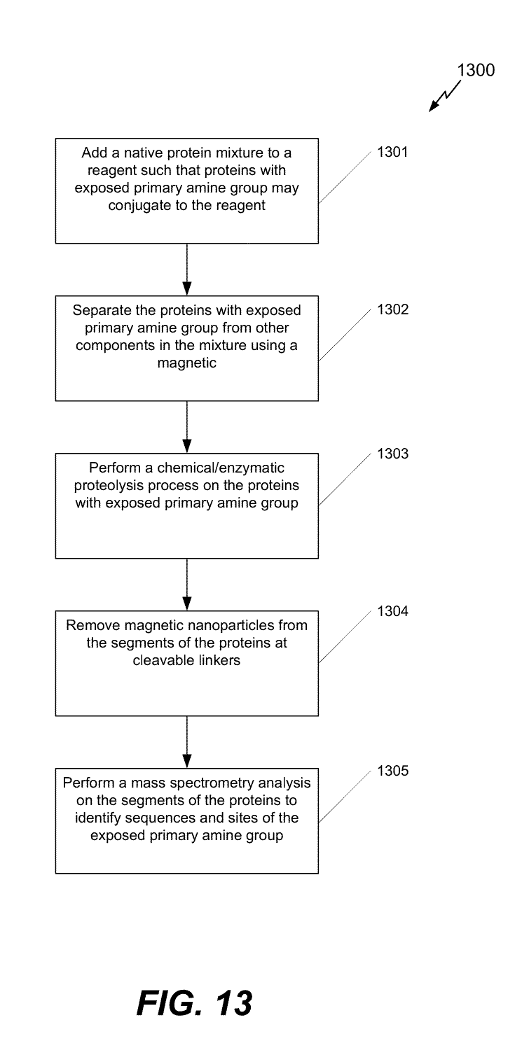

Referring to FIG. 13 is a flow chart of a particular embodiment of a method of using a reagent to identify sequences and labeled sites of a protein with exposed primary amine groups in a protein. The method may also be illustrated with reference to FIGS. 10 and 11.

At 1301, a solution that includes a native protein mixture may be added to a reagent 1001 (e.g., the reagent 100 of FIG. 1, 703 of FIG. 7, and 903 of FIG. 9) with continuous stirring. The reaction may last for a specific period of time at room temperature. Proteins with exposed primary amine groups (e.g., the protein with an exposed primary amine group 1002) may conjugate to the reagent.

At 1302, the proteins with exposed primary amine groups conjugating to the reagent (e.g., the protein with an exposed primary amine group 1003) may be separated from other components in the mixture using a magnet.

At 1303, a chemical/enzymatic proteolysis process may be performed on the proteins with the exposed primary amine groups conjugating to the reagent to form segments of the proteins (e.g., the peptide with an exposed primary amine group 1004).

At 1304, the segments of the proteins with the exposed primary amine groups conjugating to the reagent (e.g., the peptide with an exposed primary amine group conjugating to a magnetic nanoparticle 1102) may be configured to be removed from the magnetic nanoparticles at the cleavable linkers. In one embodiment, tris(2-carboxyethyl)phosphine (TCEP) 1101 may be added to the segments of the proteins with the exposed primary amine groups conjugating to the reagent. The mixture may be incubated for a specific period of time at room temperature. As a result, the segments of the proteins with the exposed primary amine groups 1103 may be separated from the magnetic nanoparticles (e.g., the magnetic nanoparticle 1104) at the cleavable linkers. The magnetic nanoparticles may be removed using a magnet.

At 1305, a mass spectrometry analysis may be performed on the segments of the proteins with the exposed primary amine groups such that sequences and sites of the segments of the proteins with the exposed primary amine groups in the proteins may be identified.

FIG. 13 thus illustrates a method of using a reagent (e.g., the reagent 100 of FIG. 1, 703 of FIG. 7, and 903 of FIG. 9) to identify sequences and sites of the exposed primary amine groups in a protein. The method may enable separation of the segments of the proteins with the exposed primary amine groups from other components. The method may also enable the segments of the proteins with the exposed primary amine groups to be separated from the magnetic nanoparticles at the cleavable linkers. The method may further enable labeling the exposed primary amine groups in aqueous solution and at/below room temperature.

The previous description of the disclosed embodiments is provided to enable a person skilled in the art to make or use the disclosed embodiments. Various modifications to these embodiments will be readily apparent to those skilled in the art, and the principles defined herein may be applied to other embodiments without departing from the scope of the disclosure. Thus, the present disclosure is not intended to be limited to the embodiments shown herein but is to be accorded the widest scope possible consistent with the principles and novel features as defined by the following claims.

What is claimed is:

1. A reagent, comprising:
a magnetic nanoparticle;
a cleavable linker; and
a primary amine reactive group,
wherein a first end of the cleavable linker conjugates to a surface of the magnetic nanoparticle,
wherein a second end of the cleavable linker conjugates to the primary amine reactive group, and
wherein the primary amine reactive group is configured to conjugate to an exposed primary amine group in a protein.

2. The reagent of claim 1, wherein size of the magnetic nanoparticle is configured such that the magnetic nanoparticle is unable to penetrate into a cell surface.

3. The reagent of claim 1, wherein the magnetic nanoparticle comprises an iron oxide (Fe₃O₄) super paramagnetic nanoparticle.

4. The reagent of claim 1, wherein the magnetic nanoparticle is coated with a coating layer prior to introducing the cleavable linker to the surface of the magnetic nanoparticle.

5. The reagent of claim 4, wherein the coating layer is configured to perform one or more functions: preventing the magnetic nanoparticle from oxidation, preventing an interaction between the magnetic nanoparticle and a protein, and facilitating surface modification by a functional group.

6. The reagent of claim 4, wherein the coating layer comprises a silica (SiO₂) coating layer.

7. The reagent of claim 1, wherein the cleavable linker comprises a disulfide bond cleavable linker, a tertiary carbamate cleavable linker, or a beta keto ester cleavable linker.

8. The reagent of claim 1, wherein the primary amine reactive group comprises a N-hydroxysuccinimidyl (NHS) ester functional group or a sulfo-NHS ester functional group.

9. A method, comprising:
forming a reagent for identifying sequences and sites of exposed primary amine groups in a protein, comprising:
forming a cleavable linker on a surface of a magnetic nanoparticle; and
conjugating the magnetic nanoparticle to a primary amine reactive group,
wherein a first end of the cleavable linker conjugates to the surface of the magnetic nanoparticle,
wherein a second end of the cleavable linker conjugates to the primary amine reactive group,
wherein the primary amine reactive group is configured to conjugate to exposed primary amine groups in the protein, and
wherein the reagent comprises the magnetic nanoparticle, the cleavable linker, and the primary amine reactive group.

10. The method of claim 9, further comprising coating the magnetic nanoparticle with a coating layer prior to forming the cleavable linker.

11. The method of claim 10, wherein the coating layer is configured to perform one or more functions: preventing the magnetic nanoparticle from oxidation, preventing an interaction between the magnetic nanoparticle and a protein, and facilitating surface modification by a functional group, and wherein the coating layer comprises a SiO₂ coating layer.

12. The method of claim 9, wherein size of the magnetic nanoparticle is configured such that the magnetic nanoparticle is unable to penetrate into a cell surface.

13. The method of claim 9, wherein the cleavable linker comprises a disulfide cleavable linker, a tertiary carbamate cleavable linker, or a beta keto ester cleavable linker.

14. The method of claim 9, wherein the primary amine reactive group comprises a NHS ester functional group or a sulfo-NHS ester functional group.

15. A method, comprising:
using a reagent to identify sequences and sites of exposed primary amine groups in proteins, wherein the reagent comprises magnetic nanoparticles, cleavable linkers, and primary amine reactive groups, wherein first ends of the cleavable linkers conjugate to surfaces of the magnetic nanoparticles, and wherein second ends of the cleavable linkers conjugate to the primary amine reactive groups, comprising:
adding the reagent to a native protein mixture such that the magnetic nanoparticles conjugate to the proteins with the exposed primary amine groups, wherein the native protein mixture comprises the proteins with the exposed primary amine groups;
separating the proteins with the exposed primary amine groups from other components of the native protein mixture under a magnetic field;
removing the magnetic nanoparticles from the proteins with the exposed primary amine groups at the cleavable linkers; and
identifying sequences and/or sites of the exposed primary amine groups in the proteins.

16. The method of claim 15, wherein the magnetic nanoparticles comprise iron oxide (Fe₃O₄) super paramagnetic nanoparticles.

17. The method of claim 15, wherein the magnetic nanoparticles are coated with a coating layer prior to introducing the cleavable linkers to the surfaces of the magnetic nanoparticles, and wherein the coating layer is configured to perform one or more functions: preventing the magnetic nanoparticle from oxidation, preventing an interaction between the magnetic nanoparticle and a protein, and facilitating surface modification by a functional group.

18. The method of claim 15, wherein sizes of the magnetic nanoparticles are configured such that the magnetic nanoparticles are unable to penetrate into cell surfaces.

19. The method of claim 15, wherein the cleavable linkers comprise disulfide bond cleavable linkers, tertiary carbamate cleavable linkers, or beta keto ester cleavable linkers.

20. The method of claim 15, wherein the primary amine reactive groups comprise NHS ester functional groups or sulfo-NHS ester functional groups.
